(19) United States
(12) Patent Application Publication
(10) Pub. No.: US 2026/0273194 A1
(43) Pub. Date: Sep. 17, 2026

(54) ADD-ON DEVICE FOR AN INJECTION DEVICE

(71) Applicant: SANOFI, Paris (FR)

(72) Inventors: Michael HELMER, Frankfurt am Main (DE); Michael SCHABBACH, Frankfurt am Main (DE)

(21) Appl. No.: 19/469,204

(22) PCT Filed: Mar. 22, 2024
(86) PCT No.: PCT/EP2024/057756

§ 371 Date: Sep. 25, 2025

(30) Foreign Application Priority Data
Mar. 28, 2023 (EP) 23315062.2

(51) Int. Cl. A61M 5/315; A61M 5/31

(52) U.S. Cl. CPC A61M 5/31566; A61M 5/3129; A61M 5/31515; A61M 5/3158; A61M 2005/3142; A61M 2202/0007; A61M 2205/3561; A61M 2205/3592; A61M 2205/505; A61M 2205/60

(57) ABSTRACT

The present disclosure relates to an add-on device for an injection device. The injection device comprises a barrel filled with a medicament and sealed by a stopper, which is movable in a distal direction to expelling a dose. A machine-readable identifier is attached to or integrated into the barrel or a housing of the injection device. The add-on device includes a body, a fastening structure connected to or integrated into the body and configured to detachably connect to the stopper, an electronic module inside the body. The electronic module comprising a reading unit operable to read information stored in or provided by the machine-readable identifier. When connected to the stopper, the body and the fastening structure are configured to transfer a user-exertable driving force onto the stopper to induce a movement of the stopper relative to the barrel.

### CROSS-REFERENCE TO RELATED APPLICATIONS

The present application is the national stage entry of International Patent Application No. PCT/EP 2024/057756, filed on Mar. 22, 2024, and claims priority to European Application No. EP 23315062.2, filed on Mar. 28, 2023, the disclosures of which are incorporated herein by reference.

### TECHNICAL FIELD

The present disclosure relates to auxiliary devices or add-on devices for use with injection devices, such as syringes, safety syringes or pen-type injectors. In a further aspect the present disclosure relates to an injection system comprising an injection device and an add-on device. In a further aspect the disclosure relates to a method of monitoring use of an injection device or injection system for injecting a medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices or auxiliary device that are generally configured for use with injection devices and which offer an electronic detection and monitoring of single or repeated dose injection procedures.

Add-on devices or auxiliary devices can be detachably connected to an injection device. An add-on device typically comprises a sensor, detector or detector arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

With particular types of injection devices, such as prefilled syringes or prefilled safety-syringes use and user acceptance of auxiliary devices or add-on devices is quite challenging, since prefilled syringes or prefilled safety-syringes may be intended for a single use. Insofar, detachably connecting or coupling of the add-on device with the respective injection device should be rather simple and failure safe.

It would be hence desirable to provide an add-on device particularly configured for use with prefilled syringes or safety syringes and which offers enhanced functionalities in terms of detection of completion of dose injection and recording of medicament related data. At the same time, the design and structure of the add-on device should be rather simple and should offer a cost-efficient production. It should also provoke a high level of user acceptance. Also, handling and use of the add-on device should be rather easy, intuitive and straightforward.

### SUMMARY

In one aspect there is provided an add-on device for an injection device. The injection device comprises a barrel filled with a medicament and sealed by a stopper. The stopper is movable relative to the barrel in a distal direction for expelling a dose of the medicament. The injection device comprises a machine-readable identifier, e.g. in form of an electronic identifier, which is attached to or integrated into one of the barrel and a housing of the injection device. The housing of the injection device may be provided optionally and accommodates the barrel filled with the medicament. The machine-readable identifier may be fixedly attached, i.e. non-movably attached, to one of the barrel and the housing.

The add-on device comprises a body and a fastening structure connected to the body or integrated into the body. The fastening structure is configured to detachably connect or to detachably fasten to the stopper of the injection device. The add-on device further comprises an electronic module inside the body. The electronic module comprises a reading unit operable to read information stored in or provided by the machine-readable identifier.

When connected to the stopper the body and the fastening structure are configured to transfer a user-exertable driving force onto the stopper to induce a movement of the stopper relative to the barrel.

With some examples and when the body is connected to the stopper the body and the stopper are in a thrust transferring configuration. This means, that a user-exertable driving force applied to the body is transferred to the stopper to induce a respective movement of the stopper relative to the barrel.

With some examples the connection between the stopper and the body is rigid in compression. Here, a user may apply a distally directed thrust or pressure onto the body of the add-on device, which due to the connection with the stopper unalterably transfers into a respective movement of the stopper relative to the barrel.

With some examples the add-on device is configured for attachment to or for an assembly in a force transmission path or drive train, which is operable to transfer an injection force, which is exerted or provided by a user, into a respective movement of the stopper relative to the barrel for expelling a dose of the medicament from the barrel.

With some examples the barrel of the injection device comprises an outlet at its distal end. The stopper may close or seal the barrel towards an opposite proximal end of the barrel. By way of the body and its mechanical connection with the stopper the body, when connected to the stopper, is operable to transfer a distally directed dispensing force onto the stopper for moving the stopper in distal direction for the purpose of dose expelling through the outlet of the barrel.

The outlet of the barrel is typically in fluid communication with an injection needle. The injection needle may be attached or integrated into the outlet of the barrel.

With some examples the fastening structure provides a direct or an indirect connection between the body of the add-on device and the stopper of the injection device. With some examples the stopper of the injection device or the injection device may comprise an elongated plunger, which is e.g. depressible by a user in distal direction to move the stopper towards the distal direction for dose expelling.

With other examples the stopper is engageable with a separate plunger. The plunger may be separately provided in addition to the injection device and a user may have to establish a connection between the plunger and the stopper. Here, the fastening structure of the add-on device is either implemented in or at the separate plunger. The fastening structure and/or the separate plunger may be a part of the add-on device, which comprises such a plunger. Hence, an elongated plunger configured for an attachment to the stopper or configured to abut with the stopper of the injection device is sized to enter the barrel from a proximal direction towards the distal direction in order to transfer a respective driving or dispensing force onto the stopper.

Accordingly, and with some examples and when the fastening structure is integrated into the body the body itself or a portion thereof provided with the fastening structure is detachably connectable with the stopper of the injection device. With other examples the fastening structure may provide an indirect mechanical coupling or connection between the add-on device and the stopper of the injection device.

With some examples there may be provided a separate plunger, e.g. in form of an elongated rod to engage with a proximal thrust receiving face or proximal end of the stopper. Here, the fastening structure of the add-on device may be configured to be attached to the proximal end of such a separate plunger. The separate plunger may contribute to the force transmission path or drive train between the body and the stopper of the injection device.

The add-on device is of particular benefit since it provides a-twofold function. It serves to read information stored in or provided by the machine-readable identifier of the injection device. Further, the add-on device can be used as an auxiliary device in the course of injecting of a dose. The add-on device itself may be arranged or may become part of a force transmission path or of a respective drive train by way of which a user-exertable driving force can be transferred onto the stopper for expelling a dose of the medicament. This way and by arranging the add-on device in the transmission path or drive train between the stopper of the injection device and a body portion of a user, e.g. a finger of the user, which is operable to exert a respective driving force, the add-on device may provide further functionalities in terms of recording or detecting of dosing or injection events, such as the start and/or the completion of a dose injection procedure.

With a further example the add-on device comprises a thrust receiving face, which is operable to receive the user-exertable driving force. The thrust receiving face is fastened to the body of the add-on device. It may be integrated into the body of the add-on device.

The thrust receiving face may be provided at a proximal end of the add-on device. It may constitute a proximal end face of the add-on device. Typically, the thrust receiving face of the add-on device is directly attachable and/or depressible by a user for injecting of a dose or to trigger a dose injection procedure. With some examples the thrust receiving face may be provided by a cover, button or cap the add-on device, which button, cover or cap may be displaceable relative to a housing of the add-one device or relative to the body of the add-on device. Hence, the thrust receiving face may be integrated into a dispensing button or operating button of the add-on device, which is depressible by a user to e.g. activate, to actuate or to control operation of the add-on device.

With a further example the electronic module of the add-on device comprises a processor. The electronic module and/or the processor is operable to detect a completion of an injection conducted by the injection device. There may be numerous examples of how to implement an end of the injection completion functionality into the add-on device. Nonetheless and irrespective of a specific dose completion detection provided by one or both of the processor or the electronic module, the add-on device may automatically track or detect when the dose injection process has been completed. By way of the electronic module, which is operable to read information stored in or provided by the machine-readable identifier, the respective information obtainable from the machine-readable identifier can be paired or combined with a dosing information, such as a date and/or a time at which the respective dose of the medicament has been injected.

According to a further example the electronic module comprises a clock connected to the processor and operable to provide a clock signal. The electronic module is operable to buffer or to store the clock signal or a timestamp derived therefrom when detecting the completion of the injection. In this way, medicament related information as obtainable by reading of the machine-readable identifier of the injection device and corresponding dosing information, i.e. information being indicative of a date and/or time at which the dose of the medicament has been injected, can be combined and e.g. stored in a memory of the add-on device.

In this way there can be provided a dosing history when the user uses the add-on device for multiple dose injection procedures. With injection devices only providing a single dose of the medicament the user is prompted to disconnect the add-on device from the used injection device and to reconnect the add-on device with another injection device, which can be used for a subsequent dose injection procedure.

According to a further example the electronic module comprises a memory connected to the processor. The processor is configured to at least one of storing data in the memory and reading data from the memory. The memory may be implemented as a digital memory. It may comprise a volatile or a non-volatile memory. By way of the memory data being indicative of a dosing procedure and/or data obtained or derived from the machine-readable identifier can be stored in the memory either separately or in a combined manner. Providing of a memory allows to collect an amount of data, which is indicative of numerous dosing procedures and/or of numerous medicament doses injected by the injection device or by a number of injection devices.

With some examples the machine-readable identifier comprises one of an electronic identifier and a visible or optically readable identifier. When implemented as an electronic identifier the machine-readable identifier comprises a wireless tag, such as a RFID, a NFC tag or any other short range or near field communication unit. Information stored in or provided by the machine-readable identifier may include medicament related information, electronic information or data, such as a name of the medicament, a name of the pharmaceutical substance of the medicament, a concentration of the medicament, a batch number, a LOT number, a manufacturing date, a manufacturing site or manufacturing location, an expiry date and/or a temperature, at which the medicament should be stored or has been stored.

With a further example the processor of the electronic module is connected to the reading unit and is operable to process electronic signals from the reading unit to detect the completion of the injection. Here, the reading unit, which is originally capable to read information stored in or provided by the machine-readable identifier of the injection device, is further configured to detect the completion of the dose injection procedure. Insofar, the reading unit and its interaction with the machine-readable identifier provides a two-fold function. On the one hand it serves to read information stored in or provided by the machine-readable identifier. On the other hand, the interaction between the machine-readable identifier and the reading unit of the add-on device enables the detection of the completion of a dose injection procedure. Insofar, and by way of the interaction between the reading unit and the machine-readable identifier all information necessary to store and/or to provide a dosing history being indicative of a specific medicament and the time or date when the specific medicament has been injected can be obtained or derived exclusively from the reading unit.

With further examples the processor, when connected to the reading unit, may be also operable to detect the beginning of a dose injection procedure. Accordingly, and by way of the interaction with the machine-readable identifier of the injection device and the reading unit of the electronic module of the add-on device the process of dose injection can be monitored rather precisely.

According to a further example the add-on device comprises an elongated plunger extension comprising an elongated rod extending in a longitudinal direction. The plunger extension and hence the elongated rod is attached to or is integral with the body and protrudes in the distal direction from a bottom or flange portion of the body. The elongated plunger extension further comprises the fastening structure at a distal end section to connect or to abut with the stopper. The fastening structure may be provided at the distal end of the elongated rod of the plunger extension. The fastening structure may form or constitute the distal end or distal end section of the elongated rod and hence of the elongated plunger extension.

The elongated rod and/or the elongated plunger extension is a mechanically rigid component. It is stiff or rigid at least with regards to the longitudinal direction. The elongated plunger extension or elongated rod is operable to provide a force transmission from the bottom or flange portion of the body of the add-on device to the stopper of the injection device. With some examples the elongated plunger extension may substitute a separate plunger of an injection device, which is originally provided to allow a user to exert a distally directed pressure or driving force onto the stopper for driving the same in distal direction relative to the barrel of the injection device.

With some examples the elongated plunger extension may be detachably connected to the bottom or to the flange portion of the body. The body of the add-on device may comprise a cylindrical shape. It may comprise a cylindrical sidewall closed towards a distal direction by a rather planar-shaped bottom or flange portion. The sidewall of the body may be closed towards a proximal end by the thrust receiving face. The thrust receiving face may be provided on a cover or the cover portion sealing or closing the proximal end of the sidewall of the body towards the proximal direction. The cover may be movable with respect to the longitudinal direction relative to the sidewall of the body. Here, the cover may substitute or mimic a dose button of an injection device. With some examples the body of the add-on device may comprise a receptacle towards its distal end. Here, the bottom or flange portion as provided at or near the distal end of the sidewall of the body may be arranged slightly recessed with respect to the distal end of the sidewall so as to form or to provide the receptacle.

The receptacle may be sized to engage or to receive a plunger flange of a syringe plunger. Here, the fastening structure of the add-on device may be provided at a distal end of the sidewall of the body, which may be provided with a fastening structure, such as a receptacle, to engage with a plunger flange of a plunger of e.g. a prefilled syringe or prefilled safety syringe.

With further examples the elongated plunger extension may be detachably connected to the bottom or flange portion of the body. When attached to the body the plunger extension protrudes distally from the body and provides a kind of a plunger in order to transfer a user-exerted driving force onto the stopper of the injection device. The distal end of the plunger extension then connects to the stopper.

With a further example the reading unit is a wireless reading unit comprising an antenna on or inside the elongated plunger extension. The antenna may be configured for wireless signal transmission and/or for wireless signal exchange with the machine-readable identifier, which may be then implemented as an electronic identifier, such as a wireless tag.

With further examples the reading unit is or comprises an optical reader, which is operable to capture optical signals emitted by or reflected from the machine-readable identifier. Here, the machine-readable identifier may be provided with a machine-readable visual code, which is readable by the optical reader or detector.

With other examples, the machine-readable identifier may comprise a magnetically encoded identifier. Here, the reading unit comprises a corresponding magnetic reading device.

By arranging the antenna on or in the elongated plunger extension it can be provided, that the antenna and hence the wireless reading unit becomes operable to to exchange wireless signals with the electronic identifier of the machine-readable identifier only or exclusively when the stopper has been moved in the distal direction by the elongated plunger extension and reaches a predefined position relative to the barrel and hence relative to the machine-readable identifier.

With some examples the transmission range between the machine-readable identifier and the reading unit of the electronic module is smaller than the longitudinal extension of the barrel of the add-on device. It may be shorter than 50% of the longitudinal extension of the barrel. With further examples the transmission range between the machine-readable identifier, e.g. the electronic identifier and the reading unit or antenna of the add-on device is less than 30% of the elongation of the barrel, less than 25% of the elongation of the barrel or even less than 20% of the elongation of the barrel. With some examples, the machine-readable identifier is a passive electronic identifier and the transmission range of the reading unit is less than 50%, less than 30%, less than 25% or less than 20% of the elongation of the barrel.

In this way and when the injection device is in an initial configuration, in which the stopper is positioned at a well-defined longitudinal distance from the machine-readable identifier it can be provided that reading of the information stored in or provided by the electronic identifier or machine-readable identifier by the reading unit is effectively disabled. For enabling a readout of the information stored in or provided by the machine-readable identifier it is then required to bring the reading unit in close vicinity to the machine-readable identifier. When the reading unit, an antenna or some other signal receiving component of the reading unit is positioned on or inside the elongated plunger extension a longitudinal distance between the machine-readable identifier and the reading unit can be effectively decreased during the process of injecting of the dose, namely while the add-on device with the elongated plunger extension is subject to a distally directed displacement relative to the barrel, thereby urging the stopper in distal direction for dispensing of the dose.

When reaching an end of dose configuration, the machine-readable identifier, which is typically fixed on the barrel, comes or gets into the transmission range of the reading unit, thereby allowing or enabling a readout of respective information stored in or provided by the machine-readable identifier.

With a further example the antenna or the like signal receiving unit of the reading unit is located or arranged at or near the distal end section of the elongated plunger extension. Accordingly, the machine-readable identifier, e.g. in form of a wireless antenna is arranged at a longitudinal position of the barrel or housing, which overlaps with the position of the stopper when the stopper has reached an end of dose position or when the injection device is in an end of dose configuration. In this way it can be provided that the antenna longitudinally overlaps with the machine-readable identifier only after the injection process has started and when the stopper has moved from a proximal stop position towards the distal end position.

With further examples an at least partial geometric overlapping of the antenna and the machine-readable identification will take place only or shortly before an end of dose configuration of the injection device has been reached. In this way, reading of the machine-readable identifier will be enabled only at the end of a complete dose injection procedure.

With other examples, the machine-readable identifier is arranged on the barrel in such a way, that reading of the information stored in or provided by the electronic identifier will be enabled as soon as the stopper has moved a predefined distance from a well-defined initial position towards the distal direction and has reached a well-defined intermediate position.

With a further example of the add-on device, the reading unit is inoperable to read the information stored in or provided by the electronic identifier when and as long as the electronic identifier is outside a transmission range of the reading unit. With some examples, the transmission range of the reading unit is comparatively small. It may be smaller than 5 cm, 3 cm, 2 cm or even smaller than 1 cm. In this way and as long as the injection device with the add-on device attached thereto is in an initial configuration, in which the stopper and hence the antenna of the wireless reading unit are located at a longitudinal distance from each other which exceeds the transmission range, a readout of the information stored in or provided by the electronic identifier is effectively disabled. Such a read-out is only enabled during or after completion of a dose injection, which is accompanied by a distally directed movement of the add-on device and the antenna provided in or on the elongated rod of the plunger extension.

With a further example the reading unit is operable to read the information stored in or provided by the electronic identifier when at least a section of the antenna overlaps or aligns with the electronic identifier and/or with the machine-readable identifier with respect to the longitudinal direction. When reaching a respective overlapping configuration a reading of the information stored in or provided by the electronic identifier can be provided. Enabling of the readout of the electronic identifier or of the machine-readable identifier may be further used as a trigger to record a clock signal or a timestamp derivable from the clock of the electronic module.

With some examples, the transferring of the machine-readable identifier, e.g. the electronic identifier, into the transmission range of the reading unit during or at the end of a dose injection procedure, can be used as a detector that dose injection is in progress or that the dose injection has been completed. In this way the reading unit provides a twofold function. It provides and reads information stored in or provided by the machine-readable identifier or electronic identifier of the injection device and further provides a trigger to record a timestamp being indicative of a date and/or a time when the respective medicament or dose of the respective medicament has been injected by the injection device.

With a further example the electronic module comprises an end of dose sensor arranged on or integrated into the body. The end of dose sensor is configured to detect at least one of a predefined position or movement of the body relative to the barrel or housing of the injection device. The end of dose sensor may operate individually or independently from the reading unit. It may be separately connected or coupled with the processor of the electronic module. The end of dose sensor is particularly configured to detect an end of dose configuration of the injection device, namely when the stopper has reached a well-defined end position relative to the barrel.

When the add-on device is attached to a plunger flange or when the add-on device forms a kind of a plunger flange manually depressible by a user for injecting of a dose the end of dose sensor may be operable to detect a mechanical contact between the add-on device with e.g. the barrel of the injection device. It may be further configured to measure a distance between the add-on device and the barrel of the injection device. With some examples the end of dose sensor may interact with a radially outwardly protruding flange, e.g. a finger flange of the injection device, which may be provided at or near a proximal end of the barrel or housing of the injection device. In an initial configuration and before injecting of the dose of the medicament the end of dose sensor is in a well-defined distance from a dedicated portion of the injection device. During dose injection this distance will decrease until reaching an end of dose configuration, in which the distance between the dedicated portion of the barrel or housing of the injection device and the dose sensor is minimal.

With a further example the end of dose sensor comprises one of an electromechanical switch, a capacitive sensor, a magnetic sensor or an optical sensor. By way of an electromechanical switch the end of dose sensor is operable to detect a mechanical contact with a dedicated portion, e.g. at a proximal end of the barrel or housing of the injection device. Likewise, a respective contact or a decrease of a longitudinal distance between the end of dose sensor and a dedicated portion of the injection device can be detected by at least one of a capacitive sensor and a magnetic sensor or even by an optical sensor. With a capacitive sensor and a magnetic sensor, respective electrically or magnetically encoded portions will be provided at or near a distal end of the barrel or housing of the injection device. The same may apply with an optical coding or identifier, when using an optical sensor as an end of dose sensor.

According to a further example the electronic module comprises a touch sensor connected to the processor. The touch sensor comprises a sensing surfaces. The sensing surface comprises a touch sensitive sensor segment. The touch sensitive sensor segment is operable to generate or to modify an electric touch signal when touched by a body part of a user. By making use of such a touch sensitive sensor, e.g. on the thrust receiving face of the add-on device, a user interaction between the add-on device when attached to the injection device or injection system and a body portion of the user, e.g. with a thumb, or a finger of a user's hand can be used to detect an end of dose configuration or completion of a dose injection.

A user holding the add-on device or an injection system in one hand may easily reach the touch sensitive sensor element to generate or modify the control signal. With some examples the touch sensor is attached to a proximal end of the add-on device, such that it is easily reachable e.g. by a thumb of the user while holding the add-on device attached to the injection device the palm and/or with further fingers of the same hand.

According to a further example the sensing surface of the sensor assembly comprises a number of touch sensitive sensor segments. Each of the sensor segments is operable to generate or to modify an electric touch signal when touched by the body part of the user. The sensor segments are spatially separated on the sensing surface in a non-overlapping way. They may be arranged next to each other in a regular or irregular manner. With some examples the entire sensing surface is covered and/or occupied by numerous sensor segments. The sensor segments may be of equal or of unequal size.

The sensor segments may belong to a touch sensitive matrix or may constitute a touch sensitive matrix, e.g. a one or two-dimensional array of touch sensitive segments. The touch sensitive segments may each comprise a capacitor or resistor, that is operable to generate or to modify an electrical signal in response to a physical contact, e.g. with a body part of a user. Insofar, the plurality of touch sensitive sensor segments forms or constitutes a spatially resolving touch sensitive sensor.

With further examples the touch sensitive surface or touch sensitive matrix comprises a matrix of electrically resistive elements, which change their electrical and measurable resistance when e.g. touched by a user. With other examples the touch sensitive surface or touch sensitive matrix comprises a matrix of capacitive elements operable to change their measurable electric capacitance when e.g. touched by a user.

With further examples the touch sensitive surface or touch sensitive matrix comprises a combination of electrically resistive sensor segments and capacitive sensor segments. Capacitive sensors may exhibit a rather low degree of electric power consumption.

With further examples the touch sensitive surface or touch sensitive matrix is based on surface acoustic wave technology that relies upon soundwaves. Accordingly, the touch sensitive surface or touch sensitive matrix comprises at least one pair of an acoustic wave transducer and an acoustic wave receiver.

With another example the touch sensitive surface or touch sensitive matrix comprises a number of optical sensors, such as photodetectors or photodiodes.

With further examples the touch sensitive surface or touch sensitive matrix comprises ultrasonic sensors. Optical sensors and/or ultrasonic sensors could be also implemented as fingerprint sensors being capable to distinguish a characteristic fingerprint of a first user from a characteristic fingerprint of a second user.

By providing a number of sensor segments on or across the sensing surface a spatially resolved contact sensing can be provided. Hence, the processor connected to the respective touch sensitive sensor segments is operable to identify those sensor segments that generate or modify an electric touch signal in response to a mechanical contact to the body part of the user. This way, the processor is operable to determine which section or subsection of the sensing surface is actually subject to a mechanical contact with the body part of the user. This allows to provide a spatially resolved touch sensing of the body part on the sensing surface of the sensor element.

According to a further example the processor of the sensor assembly is operable to detect and/or to process temporal variation of a plurality of electric touch signals generated or modified by numerous touch sensitive sensor segments. This way, the sensor is operable to detect and/or to provide a spatial profile as well as a spatial-temporal profile of individual touch sensitive sensor segments being touched by the body part of the user. Hence, the processor may be operable to ascertain and/or to detect a region on the sensing surface that is touched by the body part at a given time. The processor may be further operable to monitor a temporal modification or movement of such a region over time. Here, a region touched by the body part may be also regarded and/or denoted as a sensing area of the sensing surface.

According to a further example the touch sensitive sensor segment and/or some or each one of the plurality of touch sensitive sensor segments is operable to generate different electric touch signals in response to a variation of a pressure applied to the touch sensitive sensor segment. Insofar, the electric touch signals to be generated by a touch sensitive sensor segment vary with the pressure e.g. applied by a body part of a user getting in touch or getting in contact with this particular touch sensitive sensor segment.

In response to a variation of the pressure applied to the touch sensitive sensor segment the electric touch signal may differ in magnitude or amplitude. Furthermore, the electric touch signal may change its sign or may change its frequency or periodicity. In response to a varying pressure applied to the touch sensitive sensor segment the respective sensor segment is operable to vary the electric touch signal in a measurable way, i.e. in a way that is detectable or processable by the processor connected or connectable to the touch sensitive sensor segment.

With some examples numerous or all touch sensitive sensor segments of the sensing surface are operable to generate different electric touch signals in response to a variation of the pressure applied thereto.

With some examples the touch sensitive sensor segment is operable to generate at least two different electric touch signals in response to a variation of a pressure applied thereto. Per default the touch sensitive sensor segment may be operable to generate a first electric touch signal in response to a first pressure applied to the touch sensitive sensor segment. Here, a first electric touch signal may be generated if the first pressure applied to the touch sensitive sensor segment is above a first predefined threshold.

The touch sensitive sensor segment may be further operable to generate a second electric touch signal that differs in at least one of a magnitude, an amplitude, a sign or a frequency from the first electric touch signal. The second electric touch signal may be generated when the pressure applied to the touch sensitive sensor segment is at or above a second predefined threshold. Typically, the second threshold is above the first threshold.

Insofar, the first and second electric touch signals may be indicative of a rather low and a rather high pressure applied to the respective touch sensitive sensor segments, respectively.

With a further example the touch sensitive sensor segment or a plurality of sensor segments is or are operable to generate a variety of different electric touch signals. It is conceivable that the touch sensitive sensor segment is operable to generate at least three, at least four, at least five, at least six or at least eight different electric touch signals, each of which reflecting or indicating a respective first, second, third, fourth, fifth, sixth or eights pressure applied to the respective touch sensitive sensor segment.

With further examples, the touch sensitive sensor segment may be operable to generate an electric touch signal that varies gradually with a variation of the pressure applied to the respective touch sensitive sensor segment. Here, the electric touch signal may be directly representative of the applied pressure.

Depending on the number and size of touch sensitive sensor segments distributed across the sensing surface there can be provided a precise and rather detailed spatially resolved pressure profile of or across the sensing surface of the sensor element. In this way, the sensor assembly is operable to detect or to measure a spatially resolved pressure profile applied across the sensing surface by a body part of a user.

The spatially resolved pressure profile and/or a temporal variation of such a spatially resolved pressure profile may be indicative of a particular touch procedure or gesture conducted by the user of the injection device.

According to a further example the processor is operable to recognize one or to distinguish between of a plurality of predefined user gestures, such as a swiping across the sensing surface, a single tapping of the sensing surface and a twofold or multiple tapping of the sensing surface. The processor may be further operable to distinguish between a short-term tapping of the sensing surface, e.g. a tapping that lasts less than 1 second and a long-term tapping of the sensing surface, e.g. a tapping that lasts more than 1 or 2 seconds.

Furthermore, the processor may be operable to distinguish between a slight tapping and a firm or hard tapping of the sensing surface.

According to a further example the processor is operable to recognize at least one of a swipe motion of the body part across the sensing surface, a short-term tapping motion of the body part on the sensing surface, a multiple short-term tapping motion of the body part on the sensing surface, a long-term tapping motion of the body part on the sensing surface and a variable pressure applied by the body part on the sensing surface. These motions may represent a single gesture or multiple gestures conducted by the body part of the user on or with the sensing surface.

In a recognizing or sensing mode of the sensor assembly the electric touch signals obtained from the touch sensitive sensor segment(s) may be compared with the stored reference profiles. Here, the processor may conduct a best matching comparison in order to assign one of a stored reference profile to a profile generated or derived on the basis of the electric touch signals actually measured or detected. The reference profile assigned to the profile of electric touch signals actually measured may be then indicative of a gesture conducted by the body part of the user on the sensing surface. The control signal generated by the processor and transmitted to the external electronic device may be indicative of the gesture determined or detected by the processor of the sensor assembly.

With a further example the processor is operable to process electric touch signals of a number of touch sensitive sensor segments to ascertain a sensing area of the sensing surface being touched by the body part. Typically, and when using a finger as a body part to get in contact with the sensing surface of the sensor element the respective touch sensitive sensor segments will be able to detect a pressure applied by the respective body part. In this way all touch sensitive sensor segments getting in mechanical contact with the body part, e.g. during or for operation of add-on device or injection device may generate a respective electric touch signal.

By simultaneously processing the signals of the touch sensitive sensor segments the processor may provide or may ascertain a sensing area on the sensing surface that is actually in touch or in mechanical contact with the body part of the user. In this way, the processor is operable to detect, whether a middle part or a border region of the sensing surface is actually touched by the body part.

Moreover, the processor may be operable to detect or to measure a size of the sensing area. By ascertaining or determining a sensing area of the sensing surface being actually touched or being in mechanical contact with the body part of the user the sensor assembly provides a rather precise and spatially resolved monitoring of how the body part of the user touches the sensor element.

According to a further example the processor of the sensor assembly is operable to detect a variation of a size of the sensing area on the sensing surface. A variation of a size of the sensing area may result from a variation of the pressure applied by the body part onto the sensor element. Since the body part may comprise a certain elasticity and may further comprise a rather convex-shaped outside facing structure or surface, by increasing a pressure applied by the body part onto the sensor element the fraction of the body part getting in direct contact with the sensing surface may increase, e.g. due to an elastic deformation of the body part. This may lead to an increase of the size of the sensing area on the sensing surface.

A detection of a variation of the size of the sensing area during an operation of the injection device may be hence indicative for a variation of the pressure applied by the user onto the sensor element. With some examples the size variation measurable by the processor as well as a variation of the type, magnitude, amplitude, sign or frequency of the electric touch signals generated by the touch sensitive sensor segments may be processed in a combined manner. Insofar, a variation of the pressure applied by the body part to the sensor element may be detected by a variation of the size of the sensing area concurrently with a variation of the electric touch signals generated by the pressure sensitive and touch sensitive sensor segments.

Here, a variation of the pressure applied by the body part may be monitored or detected in a twofold manner. Hence, a variation of a pressure applied to the sensor element can be measured or determined in at least two different ways thereby increasing or providing a redundancy of the measurement system provided by the sensor assembly.

According to a further example the processor is operable to detect a variation of a geometry and/or to detect a variation of an orientation of the sensing area on the sensing surface. In this way, further modes of operation, during which the body part of the user is subject to a movement or change relative to the sensor element can be detected and evaluated. Variations of a geometry or orientation of the sensing area on the sensing surface, which is typically detected by a respective variation of electric touch signals generated by a number of touch sensitive sensor segments may be further indicative of particular scenarios of use of the injection device.

With a further example the sensing surface is of planar shape and is configured for fastening to the thrust receiving face. It may be configured to cover at least a portion of the thrust receiving face. The thrust receiving face may be provided by a removable or immovable cover forming or constituting a proximal end or end section of the add-on device or its body. Insofar, and when the add-on device is suitably attached or fastened to the injection device it may provide a thrust receiving face to be depressed by a user in order to move the stopper of the injection device to an end of content or end of dose position. The sensor may be operable during the dose injection process and may record the mechanical interaction between the sensing surface and the body portion of the user exerting an injection force on the thrust receiving surface of the add-on device.

With a further example the processor is operable to detect a completion of the injection conducted by the injection device by processing numerous electric touch signals provided by the touch sensor during a predefined time interval. With some examples a force applied by a user during an injection procedure may gradually increase during a user-induced movement of the stopper towards a distal end position. When reaching an end position a user may be obliged to keep the needle inside the punctured tissue during a predefined time interval. During this holding time the user typically applies a constant pressure onto the thrust receiving face. Such a temporary profile or pressure profile can be monitored and/or analyzed by the processor in order to determine if a dose injection procedure has completed. A respective profile may be also recorded not only with regard to a magnitude of a pressure but also with regard to a size of a sensing area of the sensing surface. An increase of the sensing area is typically a direct indication of an increase of a pressure applied by the respective body portion onto the sensing surface. With a pressure reduction also the size of the sensing area and hence the size of a mutual contact region of contact area between a body portion of the user and the sensing surface decreases.

With a further example the fastening structure of the add-on device is configured to detachably fix to a plunger flange of a plunger connected to or implemented integral with the stopper of the injection device. Here, the plunger flange may be provided at a proximal end of an elongated plunger. The distal end of the plunger may be either connected to the stopper or may form a stopper for expelling the medicament through the outlet of the barrel. The fastening structure of the add-on device may detachably connect, e.g. clipped on the flange portion.

With a further example the fastening structure of the add-on device comprises a receptacle configured to receive the plunger flange. The receptacle may be open towards the distal direction. With further examples the receptacle may be open towards a lateral direction. In either way, the receptacle may be sized and configured to detachably connect and to fix to the plunger flange of the injection device.

With a further example the fastening structure comprises a snap element configured to detachably engage with the plunger flange in a form fitting manner. The snap element may comprise a protrusion protruding radially inwardly from a sidewall of the receptacle as provided at or near the bottom or flange portion of the body of the add-on device.

With the fastening structure configured to detachably fix to a plunger flange of a plunger of an injection device the add-on device may be void of an elongated plunger extension or elongated rod. Here, the elongated plunger extension may be detachably connectable to the body of the add-on device. In this way, the add-on device may be used in different configurations and for differently configured injection devices. With injection devices being void of a plunger rod and comprising a stopper located inside a barrel the add-on device may be equipped with the elongated plunger extension. Here, the fastening structure is typically provided at the distal end of the plunger extension to make or to establish a mechanical contact with the stopper of the injection device.

With other examples and wherein the injection device comprises a plunger with a plunger flange located proximally from a proximal end of the barrel of the injection device the add-on device may be void of the elongated plunger extension and may comprise a fastening structure directly at a bottom or at a flange portion of the body. Here, a receptacle or the like fastening structure of the body can be directly attached to the plunger flange of the injection device. With both scenarios and when the add-on device is duly attached to the stopper of the injection device it can be used by a user to exert a distally directed pressure onto the stopper for driving the stopper in distal direction relative to the barrel for the purpose of expelling of a dose from the barrel.

With those examples of the add-on device being void of an elongated plunger extension readout of the electronic identifier may be obtained at or shortly before an end of dose configuration has been reached, in which the longitudinal distance between the add-on device and hence of the reading unit of the add-on device and the machine-readable identifier of the injection device is minimal.

It is also conceivable that the reading unit is manually triggered to read the machine-readable identifier of the injection device before the add-on device is mounted or attached to the plunger or plunger flange of the injection device. Here, a user may prompt the add-on device to read the information stored in or provided by the electronic identifier, e.g. by bringing the add-on device in close vicinity to the machine-readable identifier of the injection device. In other words, a user may arrange the add-on device and/or its reading unit in a distance to the machine-readable identifier of the injection device, which distance is smaller than the transmission range of the reading unit. Then, readout of information stored in or provided by the machine-readable identifier of the injection device can be obtained and the add-on device can be attached or fastened to the injection device thereafter.

In another aspect the present disclosure relates to an injection system. The injection system comprises an injection device and an add-on device as described above. The injection device comprises a medicament container. The medicament container comprises a barrel filled with a medicament, e.g. a liquid and hence injectable medicament. The barrel is sealed by a stopper. The stopper is movable relative to the barrel in a distal direction for expelling a dose of the medicament from the barrel. The injection device further comprises a machine-readable identifier attached to or integrated into the barrel or a housing of the injection device.

The machine-readable identifier may comprise or may constitute an electronic identifier. The electronic identifier may be implemented as a wireless tag, such as a RFID tag or NFC tag. The electronic identifier may be a passive electronic tag operable to communicate with a corresponding wireless and hence electronic reader of the add-on device. With some examples the machine-readable identifier is one of a magnetically encoded identifier and an optical or visual identifier, which is readable by an optical reader or by a respective magnetic reading device.

With some examples the injection system comprises an injection device as described above. Insofar, all features, effects and benefits as described above in connection with the add-on device and the injection device equally apply to the injection system.

With a further example the electronic identifier is arranged at or near a distal end section of the barrel. The reading unit is a wireless reading unit and comprises an antenna on or inside an elongated plunger extension protruding in the distal direction from a bottom or from a flange portion of the body, hence from a distal end portion of the body. Here, the antenna may be located at a longitudinal distance from the electronic module of the device. It may comprise a limited transmission range, so that reading of the machine-readable identifier and hence of the electronic identifier is only enabled by moving the plunger extension from a proximal start position towards a distal end position, thereby pushing or urging the stopper of the injection device towards a distal end position.

A distally directed movement of the stopper, e.g. from a proximal end position towards the distal end position is inducible and/or is controllable by the add-on device being in mechanical pressure-or thrust-transferring contact or engagement with the stopper.

With the antenna on or inside the elongated plunger extension and with the antenna e.g. located at or near a distal end or end section of the plunger extension, a longitudinally overlapping arrangement or configuration of the antenna and the machine-readable identifier may be obtained or may coincide with the stopper reaching or approaching a distal end position relative to the barrel. It may be only until then that the information stored in or provided by the machine-readable identifier becomes readable by the wireless reading unit. Typically and as described above, the add-on device is provided with a clock, which is operable to provide a clock signal on the basis of which a timestamp can be generated. Generation of the timestamp and/or use of a respective time or date indication as provided by the clock can be triggered by enabling a reading of the information stored in or provided by the machine-readable identifier, namely as the stopper as driven by the add-on device reaches or approaches a distal end position and hence an end of dose configuration.

With a further example the injection system is transferable from an initial configuration into an end of dose configuration by pushing the stopper from a proximal end position towards and into a distal end position via the add-on device. Here, the add-on device is attached to the stopper, either by way of the longitudinal or elongated plunger extension and its elongated rod or by way of a plunger of the injection device. Here the plunger of the injection device may be attached to or may be integrally shaped with the stopper configured to seal the volume of the interior of the barrel occupied by the medicament. Then, the body of the add-on device is attachable and/or fixable to a proximal end of the plunger or plunger rod of the injection device. It may be fixed to a proximally located plunger flange.

With a further example the electronic identifier is outside a transmission range of the reading unit or antenna when the injection system is in the pre-use configuration.

With a further example the electronic identifier is within the transmission range of the reading unit or antenna when the injection system is in the end of dose configuration. In this way, reading of the information stored in or provided by the electronic identifier requires a distally directed movement of the reading unit or antenna relative to the machine-readable identifier in the course of dose injection.

When reaching an end of dose configuration or when approaching the end of dose configuration reading of the information will be enabled. Read-out of information may then trigger the generation or providing of a timestamp, which may be then combined with the information gathered or obtained from the machine-readable identifier. The information together with the timestamp may be stored in a memory of the add-on device and may be transmitted to an external electronic device, such as a smartphone, a smartwatch or a tablet computer for further data analysis or precision monitoring.

In another aspect the present disclosure also relates to a method of monitoring use of an injection device. The method includes the step of providing the injection device as described above. Accordingly, the injection device comprises a barrel filled with a medicament and sealed by a stopper, typically in proximal direction. The stopper is movable relative to the barrel in a distal direction for expelling a dose of the medicament.

The injection device further comprises a machine-readable identifier attached to or integrated into the barrel or housing of the injection device.

The method includes providing of an add-on device as described above and reading information stored in or provided by the machine-readable identifier through the reading unit of the add-on device.

As described above, reading of the information stored in or provided by the machine-readable identifier may be implemented in a variety of different ways. With one example the machine-readable identifier comprises an electronic identifier, such as a wireless transceiver, e.g. in form of a passive or active RFID tag or NFC tag, which is readable by a wireless reading unit. Here, the reading unit of the add-on device comprises a respective transceiver operable to interact with the electronic identifier.

With further examples the machine-readable identifier comprises a magnetic code, which is readable by a reading unit implemented as a magnetic reading device. With another example the machine-readable identifier comprises an optical or visual code, which is readable by the reading unit comprising a respective optical reader.

With some examples reading of the information stored in or provided by the machine-readable identifier is conducted before or after attaching the add-on device to the injection device, i.e. before or after attaching or connecting the body of the add-on device directly or indirectly with the stopper of the injection device. With some examples reading the information stored in or provided by the machine-readable identifier takes place after or during the process of dose injection, i.e. when the stopper of the injection device is subject to a distally directed movement relative to the barrel or when the stopper reaches or approaches a distal end position and hence an end of dose configuration.

With a further example the add-on device is connected to the stopper in a pressure transmitting manner. Here, a fastening structure of the add-on device is either directly connected with a complementary-shaped counter fastening structure of the stopper. With some examples the fastening structure of the add-on device is operable to connect to a plunger of the injection device, wherein the plunger is connected the stopper, is integral with the stopper or is in longitudinal abutment with the stopper.

With another example the driving force is exerted onto the add-on device and is transferred onto the stopper to move the stopper in the distal direction relative to the barrel. Here and in this way, the add-on device can be used in a twofold manner. It provides a force transmission between a body portion of a user providing and exerting a pressure onto the stopper for conducting or controlling an injection procedure. Second, the add-on device provides a read-out of information or data stored in or provided by the machine-readable identifier.

With a further example the method includes a detection of a completion of an injection by the electronic module of the add-on device. Detecting a completion of the injection procedure may be conducted in different ways. With one example completion of dose injection may be detected by the reading unit, wherein reading of the machine-readable identifier is initially disabled and will be enabled during, or at the end of a dose injection procedure, e.g. when the stopper reaches or approaches an end of dose position or end of dose configuration.

With another example completion of an injection may be detected by way of a touch sensor, e.g. provided or integrated into the add-on device. With another example the completion of a dose injection procedure may be detected by a separate end of dose sensor being e.g. operable to detect a dedicated position and hence well-defined position of the add-on device relative to the barrel or housing.

With a further example the method includes recording at least one of a date or time of the completion of dose injection in a memory of the add-on device. Recording of the respective time stamp may take place concurrently with recording of information stored in or provided by the machine-readable identifier. Such information is typically indicative of the medicament stored or provided in the barrel of the injection device.

Generally, the scope of the present disclosure is defined by the content of the claims. The injection system, the auxiliary device and/or the injection device and respective methods as described herein are not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29human insulin; B30-N-myristoyl-ThrB29LysB30 human insulin; B30-N-palmitoyl-ThrB29LysB30human insulin; B29-N-(N-palmitoyl-gamma-glutamyl)-des(B30) human insulin, B29 -N-omega-carboxypentadecanoyl-gamma-L-lutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, examples of an injection system comprising an injection device and an add-on device for monitoring use of the injection device will be described in greater detail by making reference to the drawings, in which:

FIG. 1 schematically illustrates an example of an injection system comprising an add-on device and comprising an injection device in a configuration before the add-on device is connected or attached to the injection device,

FIG. 2 shows the injection system of FIG. 1 with the add-on device attached to the injection device before conducting an injection,

FIG. 3 shows the injection system of FIGS. 1 and 2 after completion of an injection,

FIG. 4 shows a further example of the injection system,

FIG. 5 shows the example of FIG. 4 at the end of injection, p FIG. 6 shows a further detail of a machine-readable identifier connected or attached to the barrel or housing of the injection device.

FIG. 7 shows a longitudinal cross-section of an injection device implemented as a safety syringe before or in the course of attaching the add-on device to the injection device,

FIG. 8 shows the example of FIG. 7 at the end of the dose injection and

FIG. 9 shows the device of FIGS. 7 and 8 after retraction of the injection needle into a housing of the injection device,

FIG. 10 schematically illustrates a numerous components of the add-on device and their assembly inside the body of the add-on device,

FIG. 11 shows another example of an add-on device during readout of information stored in or provided by a machine-readable identifier,

FIG. 12 shows an assembly of the add-on device to a plunger of an injection device,

FIG. 13 shows the injection system to communicate with an external electronic device,

FIG. 14 shows a further example of a touch sensor in a first configuration,

FIG. 15 shows the touch sensor of FIG. 14 in a second configuration,

FIG. 16 shows a further example of a touch sensor in a first configuration,

FIG. 17 shows the touch sensor of FIG. 16 in a second configuration,

FIG. 18 shows a diagram illustrating a pressure profile as measurable by the touch sensor according to FIGS. 14-17,

FIG. 19 shows a block diagram of the electronic component of the add-on device,

FIG. 20 shows a flowchart of a method of monitoring use of an injection device by making use of the add-on device, and

FIG. 21 shows another flowchart illustrating another method of monitoring use of the injection device.

### DETAILED DESCRIPTION

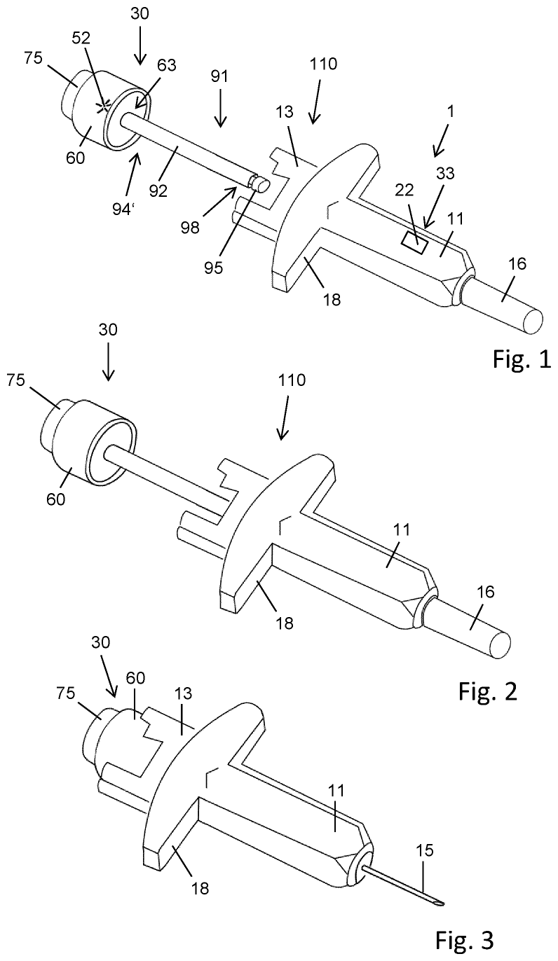

In the sequence of FIGS. 1-9 a scenario of using an injection device 1 with an add-on 30 is schematically illustrated. With the illustrated example the injection device 1 comprises a kind of a syringe, e.g. implemented as a safety syringe. The injection device 1 comprises a barrel 10 filled with a liquid injectable medicament 8. Towards a proximal longitudinal direction 3 the barrel 10 is sealed by a stopper 9, which is movable in distal direction 2 relative to the barrel 10. The barrel 10 may be of tubular shape. It may comprise an outlet 14 at its distal end. The outlet 14 may be provided with an injection needle 15 fastened to the outlet 14 of the barrel 10. Towards the proximal end or proximal direction 3 the barrel 10 terminates with a radially outwardly extending or protruding flange portion 17, which could be used as a finger grip for an index finger 6 and a middle finger 7 of a user to suitably hold the syringe while the user may use a thumb 5 of the same hand 4 to press against a plunger 20 protruding proximally from the barrel 10.

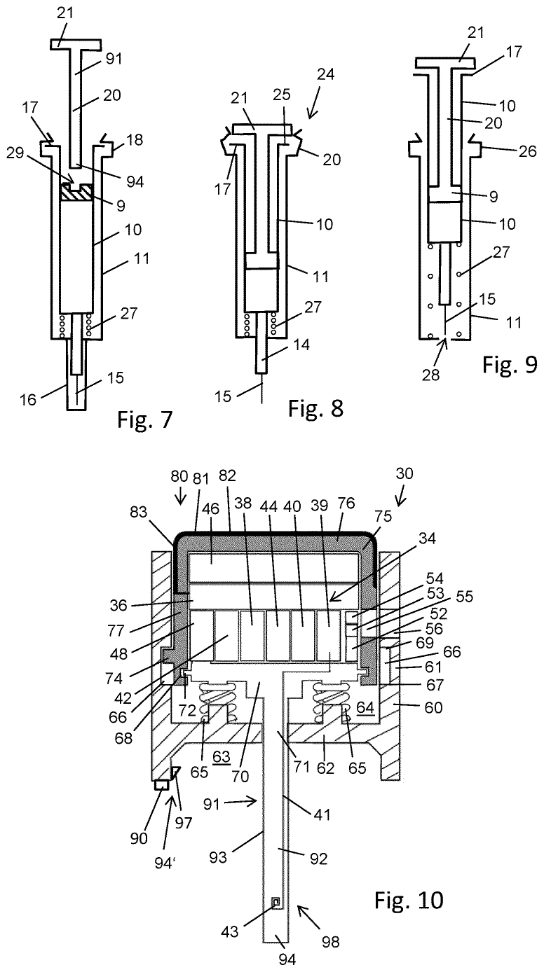

With some examples, as e.g. illustrated in FIGS. 7-9 , the stopper 9 may be integrally formed or detachably connected with a longitudinally extending plunger 20. The plunger 20 may comprise an elongated rod extending in longitudinal direction (z). With some examples the stopper 9 and the plunger 20 may be provided as separate pieces. Here, the plunger 20 may be used to exert a distally directed pressure onto the stopper 9 in order to move the stopper 9 in distal direction 2 relative to the barrel 10, thereby expelling a well-defined amount of the liquid medicament 8 through the outlet 14 and into the biological tissue when the injection needle 15 has penetrated or pierced the tissue. The stopper 9 may comprise an elastomeric material to hermetically seal the interior of the barrel 10.

The proximal end of the plunger 20 may be provided with a radially widened plunger flange 21. The plunger flange 21 is typically configured to be depressed by a thumb 5 of a user as e.g. illustrated in FIG. 10. With the example of FIGS. 1-9 the injection device 1 is implemented as a safety syringe. It is provided with a housing 11, which is of substantially elongated or tubular shape. The housing 11 is sized to accommodate the barrel 10 as well as a spring element 27. The spring element 27 is operably engaged with the housing 11 and with the barrel 10. It may be configured to induce a relative longitudinal motion between the barrel 10 and the housing 11, particularly with a completion of a dose injection procedure.

The barrel 10 may be secured against a longitudinal motion relative to the housing 11 by an interlock 24. In the example as illustrated in FIGS. 7-9 , the interlock 24 is provided at or near a proximal end of the barrel 10 or housing 11. The housing 11, which may be also of sleeve-like shape, comprises a counter latch element 26 to releasably engage with a latch element 25 as provided on the barrel 10. The latch element 25 and the counter latch element 26 are initially in an engaged configuration by way of which a longitudinal displacement of the barrel 10 relative to the housing 11 is impeded or locked.

It is only upon the plunger 10 reaching a distal end configuration, in which the plunger flange 21 engages the counter latch element 26 in such a way that the counter latch element 26 is deformed of pivoted so as to give way for the latch element 25. Once the latch element 25 is released from the counter latch element 26, mechanical energy stored in the spring element 27 can be released thus leading to a longitudinal movement or longitudinal displacement of the barrel 10 relative to the housing 11.

The outlet 14 or the needle 15 initially protruding from the distal end of the housing 11 and reaching through a through opening 28 as provided on a distal end face of the housing 11 is or are retracted into the interior of the housing 11 as illustrated in FIG. 9. Here, the injection needle 15 is in a shielded or protected configuration, in which the injection device 1 cannot be used any longer and wherein the distally protruding tip of the injection needle 15 is protected by the housing 11. The flange portion 18 may be complementary shaped to the flange portion 17. The proximal end of the flange portion 18 and/or the proximal end of the housing 11 may be further provided with a beveled edge, which is configured to engage with the distally facing side or edge of the plunger flange 21 as the plunger 20 reaches a distal end position as shown in FIG. 6. When the plunger flange 21 abuts against the beveled edge, the beveled edge and hence the counter latch element 26 is urged radially outwardly, thereby disengaging from the latch elements 25, which may be simply provided by the radially outwardly extending flange portion 17 of the barrel 10.

As further illustrated in FIGS. 1, 2 and 4, and in an initial configuration the outlet 14 and/or the injection needle 15 may be protected or covered by a separate protective cap 16. Before conducting an injection procedure, the protective cap 16 should be detached and removed from the outlet 14 or needle 15. In the presently illustrated example the counter latch element 26 may be located at or near a radially outwardly extending flange portion 18 of the housing 11.

Optionally, and as e.g. apparent from FIGS. 1-3 the barrel 10 of the syringe may be encapsulated or mounted in a syringe carrier 13, which syringe carrier 13 is operably engaged with the spring element 27. The syringe carrier 13 may provide a dedicated sliding function inside the housing 11 and may house the barrel 10 inside.

The injection device 1 is further provided with a machine-readable identifier 22. The machine-readable identifier 22 is attached to or integrated to or in one barrel 10 and the housing 11 of the injection device 1. The machine-readable identifier 22 may be implemented as an electronic identifier 33. It may comprise a wireless communication tag, such as a passive RFID tag or NFC tag. The machine-readable identifier 22 may be also implemented in form of a magnetic coating or optical coding being readable by a respective reading device, e.g. a magnetic reader or optical reader of the add-on device 30. The machine-readable identifier 22 stores and/or provides electronic information or data, such as a name of the medicament, a name of the pharmaceutical substance of the medicament, a concentration of the medicament, a batch number, a LOT number, a manufacturing date, a manufacturing site or manufacturing location, an expiry date and/or a temperature, at which the medicament should be stored or has been stored.

The electronic information or data can be read out or captured by a reading unit 39 of the add-on device 30 as will be explained below. With some examples and as e.g. illustrated in FIGS. 1, 4 and 5 the machine-readable identifier 22 is arranged at or near a distal end of the barrel 10 or housing 11. It is arranged close to the outlet 14 of the injection device 1.

As it is particularly illustrated in FIG. 6, the machine-readable identifier 22 may comprise an antenna 23 for wireless signal transmission with the reading unit 39 of the add-on device 30. The antenna 23 may be provided on a pliable substrate or layer 121. The layer 121 may comprise a plastic foil or any other pliable or bendable layer or sheet material to be wrapped around one of the barrel 10 and the housing 11 of the injection device 1.

The layer 121 may be provided with a printed electronic circuit, constituting or comprising the wireless communication tag. On a lower side of the layer 121 there may be provided a printed label layer 122. The label layer 122 may be provided with printed information, which when wrapped around the barrel 10 or housing 11 is visible by a user. Here, and when in a wrapped configuration the layer 121 is on the inside of the label layer 122, which label layer comprises a printed and visible surface on its opposite outside facing side.

On the inside of the layer 121 and around the antenna 23 there may be provided a screening or isolation layer 120, operable to screen electromagnetic radiation. The screening or isolation layer 120 may comprise a metal foil, which is insulated with regard to the electronic circuit of the machine-readable identifier 22. It may be cut-out or may be exempted around the antenna 23. The screening or isolation layer and the cut-out help to prevent an unauthorized or premature readout of data from the machine-readable identifier 22. For reading the data from the machine-readable identifier 22 it is required that a respective antenna 43 of a suitable reading unit 39 at least partially overlaps with the antenna 23 in longitudinal direction (z).

The add-on devices 30 as illustrated in detail in FIG. 10 is detachably connectable to the stopper 9 through a fastening structure 94, 94′. The add-on device 30 comprises a body 60 with a tubular-shaped sidewall 61.

Towards the distal end the sidewall 61 confines a receptacle 63, which is sized to receive a plunger flange 21. For this, the inside of the sidewall 61 may comprise one or numerous fasteners, such as elastic fastening ribs, which are configured to provide a slip free fastening of the add-on device 30 to the plunger flange 21 of a plunger 20 of a syringe as e.g. illustrated in FIG. 7.

The receptacle 63 is confined by a radially inwardly extending bottom or flange portion 62. The flange portion 62 separates the interior of the device body 60 into a distal receptacle 63 and a proximal receptacle 64. The proximal receptacle 64 is sized to accommodate a support 70 and a cover 75 to house an electronic module 34. The support 70 comprises a longitudinally extending stem 71 extending through the flange portion 62 in distal direction. The support 70 is displaceably mounted inside the receptacle 64. It is movable against the action of a return element or several return elements 65 in distal direction 2. The support 70 is connected with a cup-shaped cover 75, which protrudes in proximal direction from the sidewall 61 of the device body 60.

The cover 75 comprises a planar-shaped end face 76 facing in proximal direction 3. With some examples the end face 76 is provided with a touch sensor 80. Even the entirety of the end face 76 may be covered by the touch sensor 80. In this way, the end face 76 serves as an actuation surface to be depressed by a user, e.g. by a thumb 5 of a user. The user may apply a distally directed force onto the end face 76, thereby urging the cover 75 and the support 70 in distal direction 2 against the action of the return element 65.

Upon releasing the end face 76, the return element 75, e.g. implemented as a return spring, serves to move the support 70 and the cover 75 towards the proximal starting position as illustrated in FIG. 10. At least one of the cover 75 and the support 70 comprises a radially outwardly extending projection 74 guided in a longitudinally extending recess 66 on the inside of the sidewall 61 of the device body 60. As seen in longitudinal direction the recess 66 is confined by a proximal stop face 69 and a distal stop face 67. In the initial configuration as illustrated in FIG. 10 the projection 74 is in longitudinal abutment with the proximal stop face 69 of the recess 66.

When depressing the cover 75 in distal direction 2 against the action of the return element 65 a distal end position is reached when the projection 74 engages the distal stop face 67. By way of the projection 74 guided in the groove 66 the longitudinal movement of the support 70 and the cover 75 can be delimited within predefined margins relative to the device body 60. Here, the support 70 and/or the cover 75 may form or constitute an auxiliary trigger of the add-on device. When the cover 75 is in distal abutment with the stop face 67 of the sidewall 61 any further distally directed pressure present to the cover 75 unalterably transfers to the sidewall and hence to the body 60, which when attached to the stopper 9 then transfers into a respective distally directed movement of the stopper 9 relative to the barrel 10.

When depressed in distal direction 2 the support 70 and its stem 71 start to protrude from the flange portion 62. In either way, and when a user pressed against the cover 75 in distal direction 2, the respective driving force can be transferred either via the stem or via the sidewall 61 onto the stopper 9.

With the example of FIG. 10 the stem 71 extending distally from the flange portion 62 is prolonged and extended by a plunger extension 91 comprising elongated rod 92. The elongated rod 92 comprises a fastening structure 94 at its distal end as illustrated in FIGS. 1 and 10. The fastening structure 94 is configured to connect or to attach and/or to fasten with a complementary shaped counter fastening structure 29 as provided on a proximal face of the stopper 9. With some examples the fastening structure 94 comprises a threaded portion 95, e.g. an outer threaded portion to engage with a complementary shaped threaded portion of the counter fastening structure 29 of the stopper 9.

With other examples the plunger extension 91 may be connected, e.g. immovably connected to the body 60. It may likewise protrude in distal direction from the tubular-shaped sidewall 21 and/or from the receptacle 63.

The inside of a sidewall of the cover 75 may comprise at least one of a radial protrusion and a recess 68 to engage with a complementary-shaped radial recess or protrusion 72 of the support 70. The cup-shaped cover 75 connected or fastened to the support 70 provides a receptacle for an electronic module 34 arranged inside the hollow space formed by the support 70 and the cover 75.

The electronic module 34 may comprise an electronic circuit 35, e.g. on a printed circuit board 36. It may further comprise a transceiver 38 and a reading unit 39, a memory 40, a clock 42, a processor 44, a power source 46, a switch 48, a sensor unit 50, and signal generator 52, e.g. comprising light sources 53, 54.

A sidewall 77 of the cover 75 may comprise a window 55 aligned with a respective window 56 in the sidewall 61 of the device body 60. This way, visual signals producible by different light sources 53, 54 located inside the hollow space of the cover 75 can be perceived and visually detected from outside the add-on device 30.

The two light sources 53, 54 may belong to a visual signal generator 52, that is operable to produce or to generate visual signals of different color and/or of variable duration. The windows 55, 56 may be provided with a light pipe or with a light guiding structure. This way, the device body 60 can be protected against ingress of dust or humidity.

With another example, the signal generator 52 may be implemented as a haptic signal generator, e.g. configured to generate a perceivable vibration of the electronic module 34. With further examples the signal generator 52 may comprise an audible signal generator, configured to generate an audible sound.

In FIG. 19 there is shown a block diagram of one example of the electronic module 34 and/or of the touch sensor 80. As illustrated also in FIG. 10 the processor 44 is mounted on the printed circuit board 36, which is connected to the sensor element 81 of the touch sensor 80 substantially covering the end face 76 of the cover 75.

In general, the electronic module 34 can be mounted on the printed circuit board 36. The electronic module 34 may be configured to communicate with an external electronic device 100, as e.g. illustrated in FIG. 13. The external electronic device 100 may be implemented as a mobile electronic device. It may comprise a smartwatch, a smart phone or a tablet computer. The electronic module 34 comprises the transceiver 38, which is configured to establish or to build-up a communication link between the external electronic device 100 and the electronic module 34. A respective communication link can be realized in a wireless or wired manner.

The external electronic device 100 may be implemented as a smart phone. The external electronic device 100 comprises a housing 101 and may be implemented as a portable electronic device. The external electronic device 100 further comprises a device processor 102 and a display 104. Optionally, the external electronic device 100 comprises a speaker and/or or a haptic user interface. In addition, the external electronic device 100 comprises a communication unit 106 operable to communicate with the transceiver 38 of the add-an device 30. With some examples the first transceiver 38 comprises a local range transceiver having a transmission range of several meters or tens of meters.

The electronic module 34 may be configured to exchange data with the external electronic device 100. Data being indicative of an operation of the injection device and gathered or collected by the electronic module 34 may be transmitted to the external electronic device via the transceiver 38. The transceiver 38 may be implemented as wireless RF transceiver, e.g. as a Bluetooth transceiver or as a Bluetooth low energy (BLE) transceiver or as a WIFI transceiver. The reading unit 39 may be also implemented as a wireless RF transceiver, e.g. as a NFC transceiver. The two transceivers may distinguish with regard to their communication protocol and/or with regard to their transmission range.

The transmission range of the transceiver 38 may be larger than the transmission range of them reading unit 39. The transmission range of the transceiver 38 may in the range of several meters or tens of meters. The transceiver 38 may be implemented as a local range or short-range transceiver. The reading unit 39 may be implemented as a near field transceiver. It may comprise a transmission range less than 10 cm, less than 5 cm, 3 cm, less than 2 cm or even less than 1 cm.

With some examples the machine-readable identifier 22 may comprises or provides electronic information or data, such as a name of the medicament, a name of the pharmaceutical substance of the medicament, a concentration of the medicament, a batch number, a LOT number, a manufacturing date, a manufacturing site or manufacturing location, an expiry date and/or a temperature, at which the medicament should be stored or has been stored. The respective information or data is readable by the reading unit 39, e.g. when in close vicinity to the machine-readable identifier 22. Once the data of the machine-readable identifier 22 is read by the reading unit 39 the respective data can be further processed by the processor 44 and can be transmitted via the local range or short-range transceiver 38 2 to the external electronic device 100. In this way, the add-on device 30 may act as a range extender for reading of an electronic identifier 33 of the injection device 1.

Readout of the electronic identifier 33, which may be implemented as a near field communication tag (NFC tag), may be provided by the near field reading unit 39 of the auxiliary device 30. Information or data thus obtained can be further processed by the processor 44 and can be transmitted to the external electronic device 100 via the further transceiver 38.

The electronic module 34 comprises a memory 40 configured to store a plurality of measurement results of the touch sensor 80. The electronic module 34 further comprises a clock 42 configured to provide each of a plurality of measurement data of the sensor assembly 80 with a time index or timestamp indicating the point of time and/or date of the detection of the respective operation of the injection device 1.

The processor 44 is configured to process signals obtained from the touch sensor 80 that arise from an operation of the sensor element 81. The electronic module 34 and hence the electronic circuit 35 is further equipped with a power source 46 configured to power the processor 44 and the touch sensor 80 as well as the clock 42. The power source 46 may be implemented as a battery. The sensor assembly 80 may further comprise a switch 48 configured to switch the electronic circuit 35 on and off or to switch the electronic module 34 between an active mode and an in active mode, e.g. a sleeping mode. The switch 48 may be implemented as an electromechanical switch. It may be operable to conduct a switching operation when a user induces a movement of the cover 75 or support 70 relative to the body 60. The position of the switch 48 between the support 70 and the cover 75 is only exemplary. It may be likewise located between the support 70 and e.g. the bottom or flange portion 62 of the body 60.

The electronic module 34 may further comprise a sensor unit 50, e.g. comprising or including at least one of the touch sensor 80 and an end of dose sensor 90. In the block diagram of FIG. 19, the sensor unit 50 may represent at least one of the touch sensor 80 and the end of dose sensor 90.

The end of dose sensor 90 is operably connected with the processor 44. It is operable to detect a particular configuration of the injection device one, e.g. when reaching an end of dose configuration. The end of dose sensor 90 may be arranged on the body 60. It may be positioned in the receptacle 63 and may face towards the distal direction 2. The end of dose sensor 90 may be implemented as an electromechanical switch, getting in direct mechanical contact with the barrel 10 or housing 11 of the injection device 1 when reaching an end of dose or end of injection configuration as e.g. illustrated in FIG. 8. With some examples the end of dose sensor 90 may get in mechanical contact with one of the flanges 17 or 18.

With other examples the end of dose sensor 90 may be implemented as a magnetic or capacitive sensor operable to qualitatively or quantitatively detect or measure a distance between the body 60 of the add-on device 30 and the barrel 10 of the housing 110 of the injection device 1. With further examples the end of dose sensor 90 may be implemented as an optical sensor architect operable to qualitatively or quantitatively detect or measure the distance between the body 60 and one of the barrel 10 or housing 11.

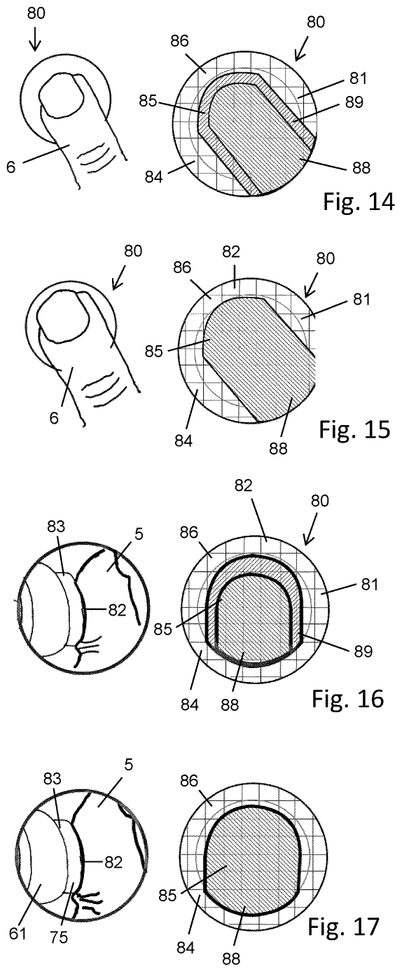

In FIGS. 14-17 examples of a touch sensor 80 are schematically illustrated. The touch sensor 80 comprises a sensor element 81 with numerous touch sensitive or pressure sensitive sensor segments 84, 85, 86 on a sensing surface 82, 83. The touch sensor 80 may comprise a regular arrangement of touch sensitive sensor segments 84, 85, 86. The individual sensor segments 84, 85, 86 may be comparable to pixels of a touch sensitive display. The sensor segments 84, 85, 86 may each comprise a capacity measuring device by way of which a mechanical contact with a body part of a user can be precisely detected.

With some examples the sensor segments 84, 85, 86 are pressure sensitive. They are hence operable to generate or to modify an electric touch signal when touched by a body part of a user. The sensor segments 84, 85, 86 may be spatially distributed across the sensing surface 82, 83 of the sensor element 81. The sensor element 81 may provide a spatially resolved detection of a sensing area 88, 89 that is actually touched by or which is in mechanical contact with the skin or body portion of a user.

With some examples the individual sensor segments 84, 85, 86 are not only touch sensitive but are also operable to generate electric touch signals being indicative of a pressure or strength of the mechanical contact with the body part of the user. Hence, the touch sensitive sensor segments 84, 85, 86 are operable to generate different electric touch signals being indicative of the magnitude of a pressure or force applied to the respective sensor segment 84, 85, 86.

Insofar, the touch sensitive sensor segments 84, 85, 86 are operable to generate electric touch signals that differ in at least one of a magnitude, an amplitude, a sign or frequency. Those differences in the electric touch signals are detectable by the processor 44 and can be evaluated so as to detect, to recognize, to characterize and/or to measure at least one of a user-induced operation of the add-on device 30 and hence of the injection device 1 when the add-on device is in a pressure transferring coupling or connection with the injection device 1.

The sensor element 81 may comprise a planar-shaped sensing surface 82 of circular shape and a tubular shaped sensing surface 83, which is located longitudinally adjacent to the outside circumference of the sensing surface 82. Insofar, and with some examples, the sensor element 81 may comprise a cup-shaped receptacle, which is sized e.g. to receive or to enclose at least a portion of the cover 75 of the add-on device 30.

The touch sensitive sensor segments 84, 85, 86 are operable to generate different electric touch signals dependent on the force level or pressure level applied to the respective sensor segments 84, 85, 86. In a scenario is illustrated in FIG. 14 the user applies a moderate or rather weak pressure onto the touch sensor 80. Accordingly, only those sensor segments 85, 86 located in a sensing area 88 may generate electric touch signals being indicative of a comparatively weak pressure. Sensor segments covered by a border region and hence by a bordering sensing area 89 surrounding or at least partially enclosing the sensing area 88 may also detect a pressure being substantially weaker than the pressure detected in the centrally located sensing area 88. Those sensor segments may generate electric touch signals being indicative of an even weaker pressure.

When a user increases the pressure applied to the touch sensor 80 as shown in FIG. 15, the sensor segments located in the sensing area 89 may experience a respective pressure increase. Accordingly, the pressure previously applied to the sensing area 89 may at least raise to the level of the pressure present in the sensing area 88 of FIG. 14. In other words, the size of the sensing area 88 being indicative of a pressure of a first level changes or increases its shape concurrently with a change of the size or shape of the sensing area 89, which decreases accordingly.

In the same or like manner the spatially resolving touch sensitive sensor 80 is operable to detect spatial and/or temporal variation of the pressure applied to the sensing surface 82. With the example of FIG. 16 a user may initially apply a comparatively weak pressure onto the sensing surface 82. Accordingly, a central sensing area 88 may reflect a pressure of a first level and a surrounding sensing area 89 may reflect a pressure of 8 second pressure level lower than the first pressure level. When the user increases a force or pressure effect on the sensing surface, by firmly pressing his thumb 5 onto the sensing surface 82 as shown in FIG. 17, due to the elasticity of the respective body portion, e.g. of the thumb 5, the region of the first pressure level, hence the central sensing area 88 extends over the surrounding sensing area 89. Accordingly, the sensing areas 88, 89 as detectable by the touch sensor 80 change their shape and/or position on the basis of which there can be derived or measured a pressure profile being indicative of a typical operation of the add-on device 30 or injected device 1.

In effect, and by way of the touch sensor 80 a pressure, a spatial pressure profile as well as temporal variations of a pressure applied onto the sensing surfaces 82, 83 can be monitored. Typical operations as applied by a user, e.g. by a thumb 5 or finger 6, 7, rolling or milling across the sensing surface 82 in the course of a dose injection procedure can be detected. On the basis of qualitatively and/or quantitatively measuring a pressure profile in the spatial and/or temporal domain the processor 44 may be operable to determine a start and/or an end of a dose injection procedure on the basis of the electric touch signals generated by the touch sensor 80.

Furthermore, there may not only be provided a spatially resolved contact profile across the one-or two-dimensional sensing surfaces 82, 83 of the sensor element 81 but the pressure profile may be also indicative of different pressure levels applied across the sensing surface(s) 82, 83.

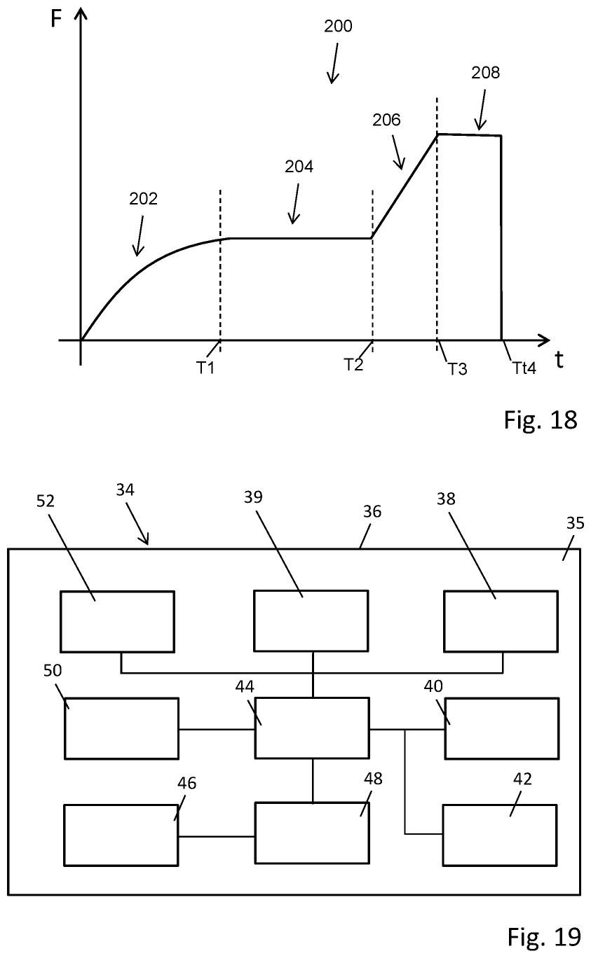

The diagram 200 of FIG. 18 is exemplary for a pressure profile, hence a pressure or force measured over time during a user conducting an injection procedure. During a first time interval 202 the user applies a distally directed pressure onto the thrust receiving face 76 of the add-on device 30 when duly attached to the stopper 9 of the injection device 1. During the first time interval 202 into the pressure increases until at time T1 the applied pressure is equal to or larger than the frictional forces between the stopper 9 and the sidewall of the barrel 10.

Accordingly and in the subsequent time interval 204, hence between times T1 and T2 the stopper 9 is subject to a constant motion in distal direction 2, thereby expelling a dose or the entirety of the medicament 8 located inside the barrel 10. At time T2 the stopper 9 reaches a distal end position. The user may then automatically apply an increased pressure since the stopper 9 does not move any further in distal direction 2. It may engage with a radially inwardly extending shoulder portion of the barrel 10 or may engage with some other longitudinally acting stop.

Accordingly, and in the time interval 206, i.e. between times T2 and T3 the measurable pressure as applied on the add-on device 30 increases to a further level. At time T3 and in a final time interval 208 lasting until the time T4 the user may apply a constant but increased pressure level to the add-on device 30 and hence to the plunger 20 or plunger extension 91 over a predefined period of time 208. This time interval 208 represents or reflects a holding time during which the needle 15 should remain in the tissue that is actually punctured. At time T4 and after lapse of the time interval 208 the user typically releases the add-on device 30 and hence the thrust receiving face 76. This results in a measurable pressure drop or force drop as detectable by the touch sensor 80. Accordingly, at time T4, the processor 44 may detect termination or completion of a dose injection procedure.

With the example of the injection system 110 as illustrated in FIG. 1 the add-on device 30 is equipped with a longitudinally extending plunger extension 91. The plunger extension 91 comprises an elongated rod 92. The diameter of the elongated rock 91 is smaller than an inside diameter of the barrel 10. As particularly illustrated in FIG. 7 the distal end of the plunger extension 91 comprises a fastening structure 94 complementary shaped or complementary configured to a counter fastening structure 29 provided at the proximal end or proximal side of the stopper 9. This way, the plunger extension 91 or rod 92 is detachably connectable with the stopper 9. With some examples the fastening structure 94 comprises a threaded portion 95, which is complementary shaped to a corresponding or complementary counter-threaded portion provided on a proximal facing side of the stopper 9.

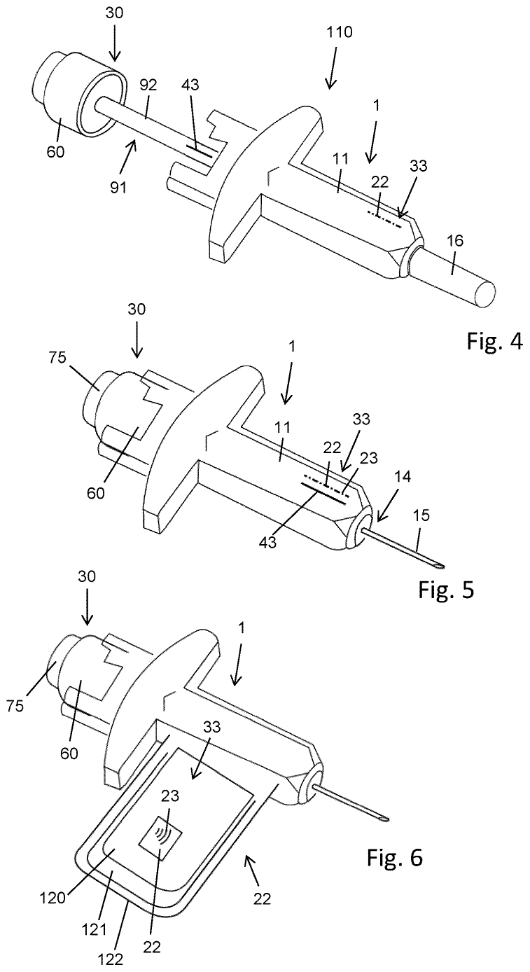

As it is further apparent from the illustration of FIGS. 4, 5 and 10 an antenna 43 of the reading unit 39 or connected with the reading unit 39 may be located on or inside the plunger extension 91. It may be also located on an outside surface 93 of the plunger extension 91. The antenna 43 may be provided at or close to a distal end section 98 of the plunger extension 91. As illustrated in FIGS. 4 and 5 the machine-readable identifier 22 of the injection device 1 may be provided at or near a distal end of the housing 11 or barrel 10. The antenna 43 is connected via a conductor 41 with the residual components of the reading unit 39, which are typically located inside the receptacle 64 and hence inside the body 60 of the add-on device 30.

In an initial configuration and prior to inject a dose as illustrated in FIG. 4, the antenna 43 located at or near the distal end 98 of the plunger extension 91 is located at a comparatively large longitudinal distance from the machine-readable identifier 22. Here, and when the machine-readable identifier 22 is implemented as a NFC tag or the like near field communication tag or identifier it may be outside the transmission range of the antenna 43 and hence out of the transmission range of the reading unit 39. Hence, in the initial configuration as shown in FIG. 4 and prior to a dose injection procedure the antenna 43 is unable and hence inoperable to read information stored in or provided by the machine-readable identifier 22.

It is only during the injection procedure or when reaching an end of dose configuration as illustrated in FIG. 5 that the antenna 43 longitudinally overlaps with the machine-readable identifier 22 or that the machine-readable identifier gets into the transmission range of the antenna 43. Insofar, the machine-readable identifier 22 becomes readable by the reading unit 39 as the antenna 43 approaches or reaches the electronic identifier 33 or machine-readable identifier 22 in longitudinal direction (z). The ability to read the information stored in or provided by the machine-readable identifier 22 may then autonomously trigger a respective readout of the information and transferring of said information to the processor 44. Concurrently with the ability to read the machine-readable identifier 22 the processor 44 may be operable to prompt or to request a timestamp from the clock 42 and to combine a respective timestamp with the information retrieved or obtained from the machine-readable identifier 22.

In this way, there can be provided a pairing between information obtained from the machine-readable identifier 22 with a timestamp, which is indicative of a time and/or date when the respective injection has been completed or conducted.

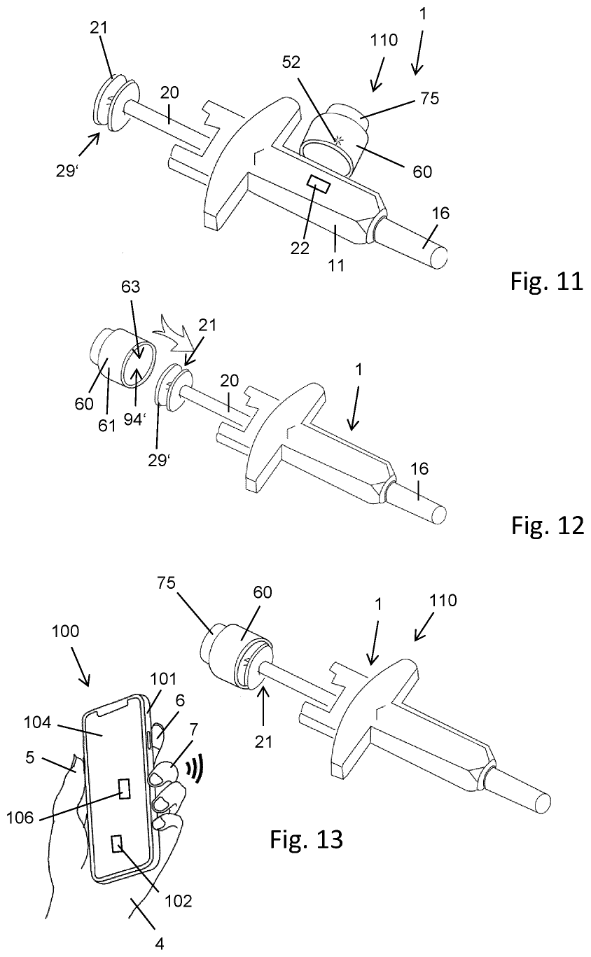

With a further example and as shown in FIGS. 11 and 12 the elongated plunger extension 91 may be provided only optional with the add-on device 30. With some examples the elongated plunger extension 91 may be detachably connected to at least one of the body 20, the flange portion 62, the bottom of the body 60 or the support 70. It may be detachably connected to the body 60 or to the add-on device. Here, and when the antenna 43 is provided in or on the elongated plunger extension 91 there may be also provided a detachable connection of a conductor 41 extending longitudinally along or through the elongated rod 92 and connected to the reading unit 91.

With a detachable longitudinal plunger extension 91 the body 60 of the add-on device 30 may be provided with a further fastening structure 94′ as illustrated in FIGS. 1 and 12. Here, the receptacle 63 confined by an inside of the sidewall 61 and the bottom or flange portion 62 may be configured to detachably fix or to detachably attach the body 60 of the add-on device 30 to the plunger flange 21 of the plunger 20 of the injection device 1. The fastening structure 94′ may be complementary shaped to a respective counter fastening structure 29′ provided on or at the plunger flange 21 of the plunger 20 of the injection device 1.

With some examples the fastening structure 94′ and the respective counter fastening structure 29′ are configured to form a friction fit. Here, an inside of the sidewall 61 of the receptacle 63 may firmly onto an outer circumference of the plunger flange 21. With another example the fastening structure 94′ and/or the counter fastening structure 29′ may comprise a snap element 97 in order to establish or to provide a form fitting engagement between the body 60 of the add-on device 30 and the plunger flange 21.

With the example as shown in FIGS. 11 and 12 the plunger 20 may be implemented as a component of the injection device 1. The plunger 20 may be connected to the stopper 9 or may be integrally formed with the stopper 9. The plunger 20 may be provided as a separate piece, which is connectable with one longitudinal end with the add-on device 30 and which is connectable with an opposite longitudinal end with the stopper 9. In either way the plunger 20 provides an indirect mechanical connection between the add-on device 30 and the stopper 9 of the injection device 1. The plunger 20 is rigid in compression at least with regards to the longitudinal direction (z). Insofar and when the add-on device 30 is attached to the proximal end of the plunger 20 it is in a force transmission path or drive train for transferring a user exertable force or pressure onto the stopper 9.

The add-on device 30 of the injection system 110 provides multiple functions. On the one hand the add-on device 30 provides an automated reading of information stored in or provided by the machine-readable identifier 22 of the injection device 1. On the other hand, the add-on device 30, when attached to the stopper 9 of the injection device 1 provides an automated injection procedure detection. The add-on device 30 may be operable to detect at least one of a start and an end of a dose injection procedure. With the example as illustrated in FIGS. 1-6 the add-on device is particularly operable to at least detect the completion of a dose injection procedure.

With the example of FIGS. 11 and 12 reading of the information stored in or provided by the machine-readable identifier 22 may be conducted prematurely, i.e. before the add-on device 30 is installed or attached to the injection device 1. The successful reading of information may be visually indicated by the signal generator 52. Moreover, the signal generator 52 may assist a user in operating or handling the add-on device 30 or the injection device 1. A variation in the duration of a signal generation, a variation of the intensity of the generated signal and/or a variation of the type of signal, e.g. with regard to signal frequency of a signal sound or signal color may further indicate to a user that a information reading procedure was successful or unsuccessful and/or that the add-on device 30 is ready to be used for transferring a user exertable force onto the stopper 9 of the injection device 1.

Detection of an end of dose configuration may be provided by enabling a readout of the machine-readable identifier 22, i.e. when the antenna 43 approaches or overlaps with the machine-readable identifier 22. With further examples an end of dose may be detected on the basis of a signal processing of signals obtainable from the touch sensor 80 and in accordance to a pressure or force profile as shown in FIG. 18. With further examples an end of dose configuration may be detected by a separate end of dose sensor 90 and/or by way of the switch 48.

The various methods or procedures of how to detect the end of a dose may be used or implemented concurrently. This way, there may be provided a redundancy in detecting an end of dose configuration of the injection device 1. Hence, information obtained from the touch sensor 80 may be concurrently processed with signals obtainable from the end of dose sensor 90. Likewise, readout of the machine-readable identifier 22, e.g. by the antenna 43 and the reading unit 39 may be processed concurrently with processing or obtaining signals from at least one of the touch sensor 80 and the end of dose sensor 90.

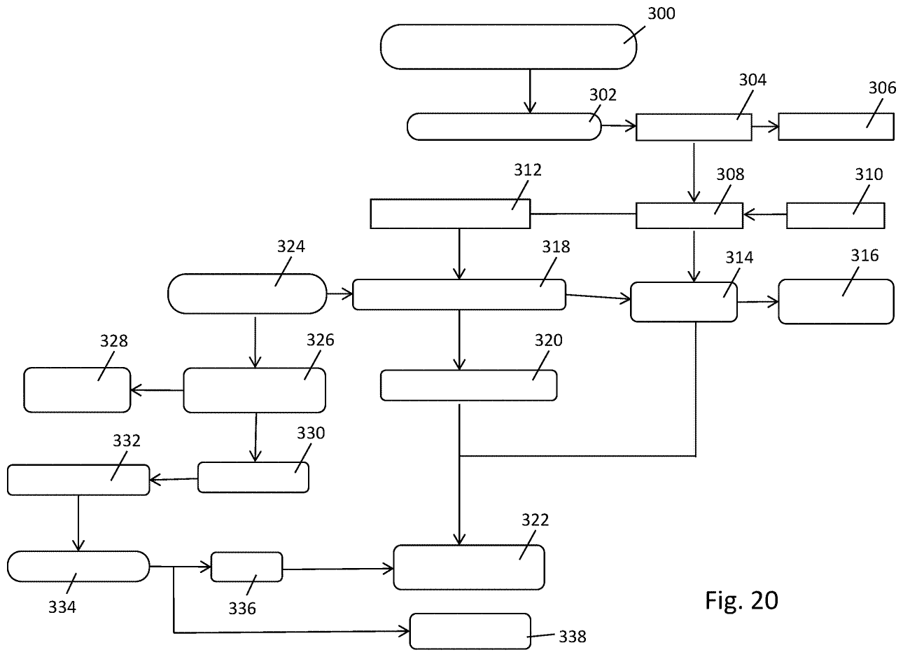

One example of a method of using the add-on device 30 with an injection device 1 is schematically illustrated in the flowchart of FIG. 20. In a first step 300 a user mounts the add-on device 30 to an injection device 1, thereby establishing a force transferring connection between the add-on device 30 and the stopper 9 of the injection device 1. In a subsequent step 302 the user simply starts the injection, e.g. by exerting a driving force onto the body 30 and/or or onto the support 70 in order to urge the add-on device 30 concurrently with the plunger 20 or plunger extension 91 in distal direction 2, thereby transferring a respective driving force onto the stopper 9.

With the movable cover 75 a user-actuatable switch 48 can be pressed or actuated such a pressure application is detected in step 304. In a subsequent step 306 and in response to a user applying pressure to the cover 75 and by moving the cover 75 in distal direction relative to the body 60 the add-on device 30 may start to provide a visual indication via the signal generator 52 thereby illustrating that the injection has started and/or that the add-on device 30 is ready to be used.

In step 310 the clock 42 provides a clock signal, which in step 308 is used to start or to control a timer. The start of the timer in step 308 trigger an activation of the reading unit 39 in step 312. In step 324 the injection device 1 has reached an end of dose configuration. Here and as a consequence the reading unit 39 in steps 312 detects the presence of the machine-readable identifier 22 and starts to read-out data from the machine-readable identifier 22. As a consequence and in step 314 the timer that has been started in step 308 is stopped. In step 316 the processor 44 conducts a plausibility check, namely if the time interval as determined by the timer matches with a typical pr predefined time interval required to inject a dose.

The information of the machine-readable in identifier is read-out in step 320. The respective information is stored in step 322 in a memory 40. Concurrently with reaching the end of dose configuration in step 324 the user is obliged to hold the injection device for a predefined time interval, e.g. 5 seconds, in the punctured tissue of the patient in step 326. At the beginning of the holding time the processor 44 may control the signal generator 52 in step 328 to accompany the holding time with the generation of a distinguishable or characteristic signal.

Here, in step 328 the signal generator 52 may produce a flashing or blinking light in a particular color, e.g. in green, so as to indicate to a user to hold the injection device until the predefined holding time has lapsed. After lapse of the holding time in step 330 in step 332 the flashing or blinking light as described with regard to step 328 turns into a constant signal generated by the signal generator in step 332.

In step 334 the user releases the button or cover 75, which then under the effect of the return element 65 returns into an initial configuration. Accordingly, the switch 48 will open and may indicate to the processor 44, that the injection procedure has successfully terminated. The opening of the switch 336 may finally trigger the storage of the data in step 322. After release of the button in step 334 the electronic module 34 may wait for a predefined time interval, e.g. a few seconds or minutes and may then switch into a sleep mode in step 338.

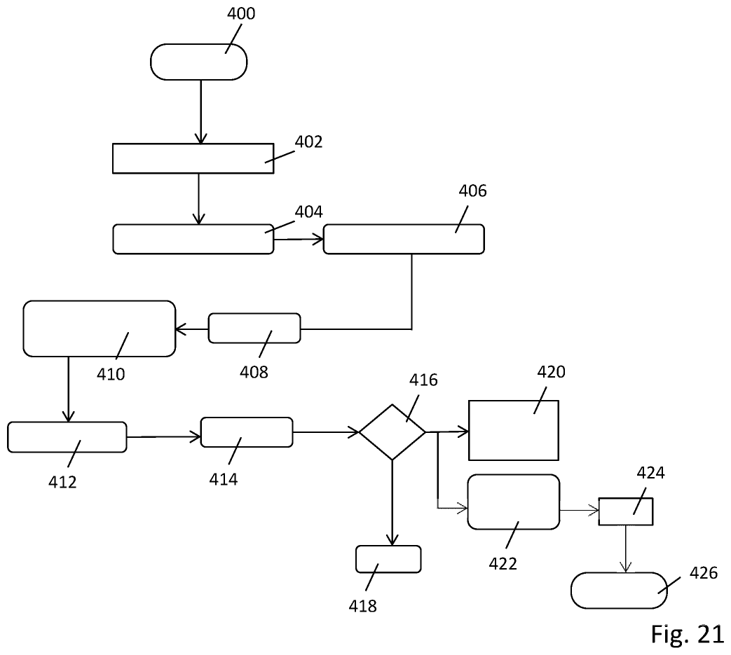

With another example and as illustrated in the flowchart of FIG. 21 detection of an end of dose configuration is provided by the end of dose sensor 90. Here the add-on device 30 may be used in a configuration as shown in FIGS. 11 and 12. Information stored in or provided by the machine-readable identifier 22 may be read out before the add-on device 30 is attached to the injection device 1. In a first step 400, the add-on device is switched from a sleep mode into an active mode. Here the reading unit 39 may be arranged in close vicinity to the machine-readable identifier 22 and respective medicament related information is transferred from the machine-readable identifier 22 to the reading unit 39. Hence, a data reading or data read-out is conducted in step 400.

The respective information obtained by the reading unit 39 is then stored in step 402 in the memory 40. In step 404, user feedback is provided to the user, e.g. by the signal generator 52 producing e.g. a flashing or blinking visual signal. Thereafter the add-on device 30 is attached to a plunger flange 21 of a plunger 20 of the injection device 1 and the patient starts to conduct the injection in step 406. Thereafter and as soon the button or cover 75 of the add-on device 30 is depressed and is thus moved relative to the body 60 the switch 48 is closed and a timer controlled by the clock 42 starts in step 408.

The information obtained from the machine-readable identifier 22 is optionally locally or temporarily stored in the memory 40 in step 410. At the end of a dose injection procedure the switch 48 may be opened in step 412 and the timer may stop in step 414. In step 416 there is conducted an assessment if the injection has been actually conducted successfully. Here, signals of at least two of the reading unit 39, the switch 48, the end of dose sensor 90 and the touch sensor 80 could be compared and evaluated in order to determine if a dose has been successfully injected and/or if a dose injection procedure has completed.

If the assessment should fail the method will be interrupted in step 418. If however, in step 416 it should be assessed that the injection has been successfully completed the respective information, i.e. the medicament related information as obtained from the machine-readable identifier 22 and a respective timestamp being indicative of the time or date of the injection are concurrently stored in step 420 and the memory 40.

Concurrently with a successful completion and with storing of the data in the memory 40 in step 422 the signal generator 52 changes its appearance and/or the signal generation. For example, a light source 53, 54 of the signal generator 52 switches to a constant green color thus indicating to a user that the injection procedure has been successfully recorded. In step 424, the electronic module 34 counts or waits for a predefined time interval before it returns into a sleep mode in step 426.

### REFERENCE NUMBERS

1 injection device
2 distal direction
3 proximal direction
4 hand
5 thumb
6 finger
7 finger
8 medicament
9 stopper
10 barrel
11 housing
12 shield
13 syringe carrier
14 outlet
15 needle
16 protective cap
17 flange portion
18 flange portion
20 plunger
21 plunger flange
22 electronic identifier
23 antenna
24 interlock
25 latch element
26 counter latch element
27 spring element
28 through opening
29 counter fastening structure
30 add-on device
34 electronic module
35 electronic circuit
36 printed circuit board
38 transceiver
39 reading unit
40 memory
41 conductor
42 clock
43 antenna
44 processor
46 power source
48 switch
50 sensor unit
52 signal generator
53 light source
54 light source
55 window
56 window
60 device body
61 sidewall
62 flange portion
63 receptacle
64 receptacle
65 return element
66 recess
67 stop face
68 recess
69 stop face
70 support
71 stem
72 protrusion
74 projection
75 cover
76 thrust receiving face
77 sidewall
79 sheet
80 touch sensor
81 sensor element
82 sensing surface
83 sensing surface
84 sensor segment
85 sensor segment
86 sensor segment
88 sensing area
89 sensing area
90 end of dose sensor
91 plunger extension
92 rod
93 surface
94 fastening structure
95 threaded portion
97 snap element
98 distal end section
100 external electronic device
101 housing
102 device processor
104 display
106 communication unit
110 injection system
120 layer
121 layer
122 layer

1-19. (canceled)

20. An add-on device for an injection device, wherein the injection device comprises a barrel filled with a medicament and sealed by a stopper, wherein the stopper is movable relative to the barrel in a distal direction for expelling a dose of the medicament and wherein the injection device comprises a machine-readable identifier attached to or integrated into the barrel or a housing of the injection device, the add-on device comprising:
a body,
a fastening structure connected to the body or integrated into the body and configured to detachably connect to the stopper,
an electronic module inside the body, the electronic module comprising a reading unit operable to read information stored in or provided by the machine-readable identifier,
wherein when connected to the stopper, the body and the fastening structure are configured to transfer a user-exertable driving force onto the stopper to induce a movement of the stopper relative to the barrel.

21. The add-on device of claim 20, wherein the electronic module comprises a processor and is operable to detect completion of an injection conducted by the injection device.

22. The add-on device of claim 21, wherein the electronic module comprises a clock connected to the processor and operable to provide a clock signal, wherein the electronic module is operable to buffer or to store the clock signal or a timestamp derived therefrom when detecting the completion of the injection.

23. The add-on device of claim 21, wherein the processor is connected to the reading unit and is operable to process electronic signals from the reading unit to detect the completion of the injection.

24. The add-on device according to claim 23, further comprising:
an elongated plunger extension comprising an elongated rod extending in a longitudinal direction (z), attached to or integral with the body and protruding in the distal direction from a bottom or flange portion of the body, wherein the elongated plunger extension comprises the fastening structure at a distal end section to connect or to abut with the stopper.

25. The add-on device according to claim 24, wherein the reading unit is a wireless reading unit comprising an antenna on or inside the elongated plunger extension.

26. The add-on device according to claim 25, wherein the reading unit is operable to read the information stored in or provided by the machine-readable identifier when at least a section of the antenna overlaps or aligns with the machine-readable identifier with respect to the longitudinal direction (z).

27. The add-on device according to claim 25, wherein the reading unit is inoperable to read the information stored in or provided by the machine-readable identifier, when and as long as the machine-readable identifier is outside a transmission range of the reading unit.

28. The add-on device according to claim 25, wherein the transmission range of the reading unit is smaller than 5 cm.

29. The add-on device according to claim 28, wherein the transmission range of the reading unit is smaller than 1 cm.

30. The add-on device according to claim 21, wherein the electronic module comprises an end of dose sensor arranged on or integrated into the body and configured to detect at least one of a predefined position or movement of the body relative to the barrel of the injection device.

31. The add-on device according to claim 21, wherein the electronic module comprises a touch sensor connected to the processor, wherein the touch sensor comprises a sensing surface, the sensing surface comprising a touch sensitive sensor segment, the touch sensitive sensor segment being operable to generate or to modify an electric touch signal when touched by a body part of a user.

32. The add-on device according to claim 31, wherein the processor is operable to detect completion of the injection conducted by the injection device by processing numerous electric touch signals provided by the touch sensor during a predefined time interval.

33. The add-on device according to claim 20, wherein the fastening structure is configured to detachably fix to a plunger flange of a plunger connected to or integral with the stopper.

34. An injection system comprising:
an injection device, the injection device comprising:
a barrel filled with a medicament and sealed by a stopper, wherein the stopper is movable relative to the barrel in a distal direction for expelling a dose of the medicament,
a machine-readable identifier attached to or integrated into the barrel or a housing of the injection device, and
an add-on device, comprising a body, a fastening structure connected to the body or integrated into the body and configured to detachably connect to the stopper, and an electronic module inside the body, the electronic module comprising a reading unit operable to read information stored in or provided by the machine-readable identifier,
wherein when connected to the stopper, the body and the fastening structure are configured to transfer a user-exertable driving force onto the stopper to induce a movement of the stopper relative to the barrel.

35. The injection system according to claim 34, wherein the machine-readable identifier is arranged at or near a distal end section of the barrel and wherein the reading unit is a wireless reading unit and comprises an antenna on or inside an elongated plunger extension protruding in the distal direction from a bottom or end portion of the body.

36. The injection system according to claim 34, wherein the injection system is transferable from a pre-use configuration into an end of dose configuration by pushing the stopper from a proximal end position towards and into a distal end position via the add-on device.

37. The injection system according to claim 36, wherein the machine-readable identifier is outside a transmission range of the reading unit or antenna when the injection system is in the pre-use configuration.

38. The injection system according to claim 34, wherein the machine-readable identifier comprises an electronic identifier, which is within a transmission range of the reading unit or antenna when the injection system is in an end of dose configuration.

39. A method of monitoring use of an injection device including the steps of:
providing the injection device, wherein the injection device comprises a barrel filled with a medicament and sealed by a stopper, wherein the stopper is movable relative to the barrel in a distal direction for expelling at least a dose of the medicament and wherein the injection device comprises a machine-readable identifier attached to or integrated into the barrel or a housing of the injection device,
providing an add-on device comprising a body, a fastening structure connected to the body or integrated into the body and configured to detachably connect to the stopper, and an electronic module inside the body, the electronic module comprising a reading unit operable to read information stored in or provided by the machine-readable identifier, wherein when connected to the stopper, the body and the fastening structure are configured to transfer a user-exertable driving force onto the stopper to induce a movement of the stopper relative to the barrel; and
reading information stored in or provided by the machine-readable identifier through the reading unit.
